(19) United States
(12) Patent Application Publication
(10) Pub. No.: US 2026/0191924 A1
(43) Pub. Date: Jul. 9, 2026

(54) METHODS AND COMPOSITIONS FOR TREATING VIRAL INFECTIONS

(71) Applicant: DST Pharma, Inc., Duluth, GA (US)

(72) Inventor: Lu Wang, Duluth, GA (US)

(21) Appl. No.: 19/554,804

(22) Filed: Mar. 3, 2026

Related U.S. Application Data
(63) Continuation of application No. 17/911,241, filed on Mar. 17, 2020.

(51) Int. Cl. A61K 36/70; A61K 31/352; A61K 31/58; A61K 31/7048; A61K 36/076; A61K 36/28; A61K 36/282; A61K 36/31; A61K 36/36; A61K 36/482; A61K 36/536; A61K 36/634; A61P 31/14

(52) U.S. Cl. CPC A61K 36/70; A61K 31/352; A61K 31/58; A61K 31/7048; A61K 36/076; A61K 36/28; A61K 36/282; A61K 36/31; A61K 36/36; A61K 36/482; A61K 36/536; A61K 36/634; A61P 31/14

(57) ABSTRACT

Nutraceutical compositions and methods of their use in treating Caliciviridae family viruses are provided. The viruses can be norovirus or sapovirus. An exemplary nutraceutical composition includes Fagopyrum dibotrys extract. Another nutraceutical composition for treating viral gastroenteritis includes 5, 7, 3′, 4′-tetrahydroxyflavan-3-ol C₄-C₈ dimers, quercetin, rutin, or a combination thereof. The nutraceutical compositions inhibit or reduce entry of Caliciviridae family viruses into intestinal epithelial cells of the subject.

### CROSS REFERENCE TO RELATED APPLICATIONS

This application is a continuation of U.S. application Ser. No. 17/911,241, filed Sep. 13, 2022, which is a national stage application under 35 U.S.C. § 371 of PCT/US2020/023157 filed Mar. 17, 2020, the contents of which are hereby incorporated by reference in their entirety.

### TECHNICAL FIELD OF THE INVENTION

This invention is generally related to nutraceutical compositions and their use to treat or prevent viral infections.

### BACKGROUND OF THE INVENTION

Noroviruses are a group of single-stranded, positive sense RNA non-enveloped viruses constituting the Norovirus genus in the family Caliciviridae. Noroviruses have been recognized as the most important cause of viral epidemic acute gastroenteritis affecting people of all ages. In the United States noroviruses cause 19 to 21 million cases of acute gastroenteritis each year, lead to 1.7 to 1.9 outpatient visits and 400,000 emergency department visits each year, and contribute to about 56,000 to 71,000 hospitalizations and 570 to 800 deaths, mostly among young children and the elderly. Norovirus is the leading cause of foodborne illness in the United States. On a worldwide basis noroviruses lead to 218,000 deaths in developing countries and 1.1 million episode of pediatric gastroenteritis in developed countries annually. Thus, norovirus associated diseases have been a heavy burden to public healthcare. Noroviruses are difficult to control owing to their widespread nature and the lack of effective vaccines and antivirals.

Transmission of these highly infectious plus-stranded RNA viruses occurs primarily through contaminated food or water, but also through person-to-person contact and exposure to objects that have been contacted with the virus. Symptoms of norovirus include fever, cramps, head and body aches, along with profound gastroenteritis, diarrhea and vomiting. Symptoms can arise gradually or abruptly and usually resolve within 48 to 72 hours. There are currently no treatments for norovirus. During an active norovirus infection it is important for the infected person to intake a sufficient amount of fluids to avoid dehydration. Intravenous fluid delivery is necessary if the infected person cannot drink enough fluids. Loss of fluid due to vomiting and diarrhea can lead to severe dehydration, and if untreated, more severe complications and even death.

Therefore, it is an object of the invention to provide compositions and methods of their use to treat norovirus infection.

It is also an object of the invention to provide nutraceutical compositions and methods of their use to prevent norovirus infection.

### SUMMARY OF THE INVENTION

Compositions and methods of their use in treating viral gastroenteritis are provided. One embodiment provides a nutraceutical composition including an amount of Fagopyrum dibotrys (F. dibotrys) extract effective to inhibit Caliciviridae family viruses such as norovirus and sapovirus. Another embodiment provides a nutraceutical composition for inhibiting Caliciviridae family viruses including 0.1 mg to 10 g of F. dibotrys extract, and optionally an excipient. In another embodiment, the nutraceutical composition optionally also include quercetin, rutin, or a combination thereof.

Another embodiment provides a nutraceutical composition for inhibiting Caliciviridae family viruses including 0.001% to 50% of 5,7,3′,4′-tetrahydroxyflavan-3-ol C₄-C₈ dimers. In one embodiment the dimers are extracted from parts of the F. dibotrys plant. The nutraceutical composition can also include 0.001% to about 50% of oligomers, isomers, and derivatives of 5,7,3′,4′-tetrahydroxyflavan-3-ol C₄-C₈ dimers.

Still another embodiment provides a nutraceutical composition including 0.001% to about 50% of 5,7,3′,4′-tetrahydroxyflavan-3-ol C₄-C₈ dimers in combination with other herbal compounds, extracts, or molecules. The herbal compounds can be milk thistle extract, Semen cassiae extract, Spica prunellae extract, Haike sapogenin, or a combination thereof.

One embodiment provides a method of inhibiting viral gastroenteritis in a subject in need thereof by administering to the subject an effective amount of the disclosed nutraceutical compositions. In certain embodiments the nutraceutical composition includes 0.1 mg to 10 g F. dibotrys extract or 0.001% to 50% of 5,7,3′,4′-tetrahydroxyflavan-3-ol C₄-C₈ dimers, and optionally quercetin, rutin, or a combination thereof.

Another embodiment provides a method of prophylactically treating viral gastroenteritis in a subject at risk of viral infection by administering to the subject one of the disclosed nutraceutical compositions. The composition can inhibit or reduce entry of Caliciviridae family viruses into intestinal epithelial cells of the subject. The compositions can be administered orally or rectally. The compositions can be administered to the subject once daily, twice daily, or three times daily. In another embodiment, the composition is administered to the subject throughout the duration of the period the subject is at risk for viral infection.

### BRIEF DESCRIPTION OF THE DRAWINGS

FIG. 1 is a bar graph showing the relative inhibition of feline calcivirus strain F9 (FCV F9) in cat kidney CRFK cells treated with various concentrations of F. dibotrys extract (XC) for 1 hour before infection with FCV F9. The X axis represents the concentration of XC extract and the Y axis represents relative inhibition of FCV F9.

FIG. 2 is a bar graph showing the relative percent inhibition of FCV F9 in CRFK cells treated with various concentrations of F. dibotrys extract (XC) concurrent with FCV F9 infection. The X axis represents the concentration of XC extract and the Y axis represents relative inhibition of FCV F9.

FIG. 3 is a bar graph showing the relative percent inhibition of FCV F9 in CRFK cells treated with various concentrations of F. dibotrys extract (XC) for 5 days after FCV F9 infection.

The X axis represents the concentration of XC extract and the Y axis represents relative inhibition of FCV F9.

FIG. 4 is a bar graph showing the percentage of CRFK cells infected with FCV F9 after treatment with 0.1% quercetin for one hour before infection with FCV F9, concurrent with FCV F9 infection, or one hour after FCV F9 infection. The X axis represents the treatment group, and the Y axis represents percentage of cells infected with FCV F9.

FIG. 5 is a bar graph showing the percentage of CRFK cells infected with FCV F9 after treatment with 0.1% rutin for one hour before infection with FCV F9, concurrent with FCV F9 infection, or one hour after FCV F9 infection. The X axis represents the treatment group, and the Y axis represents percentage of cells infected with FCV F9.

FIG. 6 is a bar graph showing the percentage of CRFK cells infected with FCV F9 after treatment with 0.1% 5, 7, 3, 4-tetrarydroxyflavon-3-ol C₄-C₈ dimers for one hour before infection with FCV F9, concurrent with FCV F9 infection, or one hour after FCV F9 infection. The X axis represents the treatment group, and the Y axis represents percentage of cells infected with FCV F9.

FIG. 7 is a bar graph showing the percentage of CRFK cells infected with FCV F9 after treatment with 0.1% quercetin and 0.1% 5, 7, 3, 4-tetrarydroxyflavon-3-ol C₄-C₈ dimers one hour after FCV F9 infection. The X axis represents the treatment group, and the Y axis represents percentage of cells infected with FCV F9.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the term “nutraceutical” refers to a pharmaceutical-grade, food-grade, and standardized nutrient, usually food or parts of food that confers health benefits. In the United States nutraceuticals are classified with dietary supplements and food additives. They can be categorized as dietary fiber, pre- and probiotics, polyunsaturated fatty acids, antioxidants, and other types of herbal and natural foods. Popular nutraceuticals include ginseng, Echinacea, green tea, glucosamine, omega-3, lutein, folic acid, and cod liver oil. Nutraceuticals are used for treating or preventing a number of diseases including arthritis, cold and cough, sleeping disorders, gastrointestinal diseases, certain cancers, osteoporosis, blood pressure, cholesterol control, pain killers, depression, and diabetes. Because nutraceuticals are naturally occurring foods or food parts, they confer fewer side effects and cost less than engineered pharmaceuticals.

As used herein, “botanical extract” refers to a concentrated solution made by extracting chemical constituents out of plant cellulose with solvent, usually a solution of alcohol and water or glycerin and water. Botanical extracts are used to maintain, restore, and improve health. As used herein, “herbal extracts” refer to a botanical extract wherein the plant is specifically an herb.

As used herein, the terms “bioactive” and “bioactive compound” refer to extra-nutritional components that are found in small quantities in foods and provide health benefits beyond the basic nutritional value of the food. Exemplary bioactive compounds include but are not limited to carotenoids, caritine, choline, coenzyme Q, dithiolthiones, flavonoids, phytosterols, phytoestrogens, glucosinolates, polyphenols, and taurine. Bioactive compounds have many beneficial properties including but not limited to antioxidant, anticarcinogenic, anti-inflammatory, and anti-microbial properties.

As used herein, “Fagopyrum” refers to a Genus of flowering plants of the family Polygonaceae, also referred to as the Buckwheat family.

As used herein, “Fagopyrum dibotrys (D. Don) Hara”, “Fagopyrum cymosum (Trev) Meisn”, and “the extract” can be used interchangeably and refer to the disclosed nutraceutical composition. F. dibotrys is a perennial herb with edible seeds and leaves that are rich in rutin. Other names for Fagopyrum dibotrys (D. Don) Hara include buckwheat, Fagopyrum esculentum, Fagopyrum leptopodum, Fagopyrum acutatum (Lehmann), and Fagopyrum megaspartanium.

As used herein, “Fagopyrum dibotrys extract” refers to a mixture of compounds present in the parts of Fagopyrum dibotrys plant (leaves, stem, seeds or root) that is extracted by water methods or by organic solvent methods.

As used herein, the terms “treat”, “treating”, “treatment” and “therapeutic use” refer to the elimination, reduction, or amelioration of one or more symptoms of a disease or disorder.

As used herein, the term “prophylactic agent” refers to an agent that can be used in the treatment of a disorder or disease prior to the detection of any symptom of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the term “effective amount” and “therapeutically effective amount” refer to the amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the terms “individual,” “subject,” and “patient” are used interchangeably, and refer to a mammal, including, but not limited to, humans, rodents, such as mice and rats, and other laboratory animals.

As used herein, “viral gastroenteritis” refers to a viral infection of the intestines that causes inflammation, swelling, and irritation to the lining of the intestines. Symptoms of viral gastroenteritis include but are not limited to fever, body aches and cramps, nausea, vomiting, diarrhea, and stomach pain. Common viruses that cause viral gastroenteritis include but are not limited to norovirus, sapovirus, rotavirus, and adenovirus.

As used herein “norovirus” are a group of single-stranded, positive sense RNA non-enveloped viruses constituting the Norovirus genus in the family Caliciviridae. Noroviruses are responsible for at least 95% of nonbacterial gastroenteritis outbreaks, and 50% of all gastroenteritis outbreaks, throughout the world. Symptoms of norovirus infection include but are not limited to fever, body aches and cramps, nausea, vomiting, diarrhea, and stomach pain.

As used herein “sapovirus” are a genetically diverse genus of single-stranded positive-sense RNA, non-enveloped viruses within the Caliciviridae family. Together with norovirus, sapoviruses are the most common cause of acute gastroenteritis in humans and other animals. Symptoms of sapovirus infection include but are not limited to fever, body aches and cramps, nausea, vomiting, diarrhea, and stomach pain.

The term, “alkyl,” as used herein, refers to the radical of saturated or unsaturated aliphatic groups, including straight-chain alkyl, alkenyl, or alkynyl groups, branched-chain alkyl, alkenyl, or alkynyl groups, cycloalkyl, cyclcoalkenyl, cycloalkynyl groups, alkyl substituted cycloalkyl, cyclcoalkenyl, or cycloalkynyl groups, and cycloalkyl substituted alkyl, alkenyl, or alkynyl groups. Unless otherwise indicated, a straight chain or branched chain alkyl has 33 or fewer carbon atoms in its backbone, preferably 20 or fewer, and more preferably 12 or fewer.

The term, “alkyl,” also includes one or more substitutions at one or more carbon atoms of the hydrocarbon radical as well as cycloalkyls, unsaturated alkyls, substituted alkyls, heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclic, aromatic or heteroaromatic moieties, —CF₃; —CN; —NCOCOCH₂CH₂; —NCOCOCHCH; —NCS; and combinations thereof.

The term “aryl” refers to a mono- or multi-cyclic aromatic radical having in the range of 3 up to 20 carbon atoms such as phenyl, naphthyl, tetrahydronapthyl, indanyl, and biphenyl.

The term, “heteroaryl,” as used herein, refers to straight or branched chain, or cyclic carbon-containing radicals, or combinations thereof, having 3 to 20 carbon atoms where one or more of the carbon atoms are replaced by heteroatoms. Suitable heteroatoms include, but are not limited to, O, N, Si, P and S, where the nitrogen, phosphorous and sulfur atoms are optionally oxidized, and the nitrogen heteroatom is optionally quarternized. One of the rings may also be aromatic. Examples of heterocyclic and heteroaromatic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

The term, “acyl” as used herein, refers to alkylcarbonyl substituents.

### II. Compositions

Nutraceutical compositions containing an effective amount of extract of Fagopyrum dibotrys (D. Don) Hara to inhibit viruses of the Caliciviridae family are provided. Caliciviridae viruses that affect humans include noroviruses and sapoviruses. The disclosed compositions are useful in the treatment of symptoms of norovirus and sapovirus infections including but not limited to diarrhea, vomiting, and stomach pain.

One embodiment provides a nutraceutical composition including 5, 7, 3′, 4′-tetrahydroxyflavan-3-ol C₄-C₈ dimers or derivatives thereof according to any one of Formula I, Formula II, or Formula III, quercetin, rutin, sapogenin, and optionally a pharmaceutically acceptable excipient. Another embodiment provides a nutraceutical composition including 1 mg to 75 mg of 5, 7, 3′, 4′-tetrahydroxyflavan-3-ol C₄-C₈ dimers or derivatives thereof according to any one of Formula I, Formula II, or Formula III, 20 mg quercetin, 20 mg rutin, and 15 mg sapogenin. The nutraceutical compositions disclosed herein can also include additional herbal compounds useful for treating disease such as synthetic catechins or polyphenols, or other plant extracts.

A. Fagopyrum dibotrys Compositions

One embodiment provides compositions of extracts of F. dibotrys. In one embodiment, the composition contains between 0.1 mg to 10 g of F. dibotrys extract. In some embodiments the compositions contain 0.1 mg, 0.5 mg, 1 mg, 5 mg, 10 mg, 20 mg, 30 mg, 40 mg, 50 mg, 60 mg, 70 mg, 80 mg, 90 mg, 100 mg, 500 mg, 1 g, 5 g, or 10 g of F. dibotrys extract. The preferred dose of extract (100:1) is 1.5 grams/day in dry form (pills or capsules). Oral formulation may include starch and other regular inactive ingredients. If only the active ingredient is considered, the preferred dose is 75 mg/day.

In some embodiments, the F. dibotrys extract is obtained from the roots of the plant. In another embodiment, the extract is obtained from the seeds of the plant. In yet another embodiment, the extract is obtained from the leaves of the plant or the flowers of the plant.

### B. Active Components

i. Flavonoid Compositions

In some embodiments, the active component of the F. dibotrys extract is 5, 7, 3′, 4′-tetrahydroxyflavan-3-ol C₄-C₈ dimers, Formula I. 5, 7, 3′, 4′-tetrahydroxyflavan-3-ol is a flavan-3-ol, a major subclass of flavonoid present in many plants. Flavonoids, including flavan-3-ols, have antioxidant, anticarcinogenic, cardiopreventative, antimicrobial, anti-viral, and neuroprotective properties.

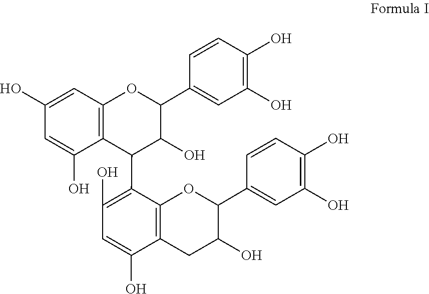 [SMILES: Oc1cc(O)c2c(c1)OC(c1ccc(O)c(O)c1)C(O)C2c1c(O)cc(O)c2c1OC(c1ccc(O)c(O)c1)C(O)C2]

In some embodiments the active component is a derivative of 5, 7, 3′, 4′-tetrahydroxyflavan-3-ol C₄-C₈ dimers according to Formula II, or a pharmaceutically relevant enantiomer, salt, or solvate thereof. Formula II is as follows:

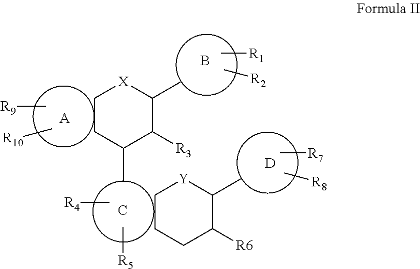

wherein:
rings A, B, C, and D are independently aryl or herteroaryl mono- or bicycylc ring systems containing zero or more nitrogen atoms including but not limited to phenyl, pryridine, pyrimidine, pyridazine, pyrazine, triazine, quinolone, quinazoline, isoquinoline, naphthalene, naphthyridine, indole, isoindole, cinnoline, phthalazine, quinoxaline, pteridine, purine, and benzimidazole;
R₁, R₂, R₃, R₄, R₅, R₆, R₇, R₈, R₉ and Rio are independently selected from —H, —F, —Cl, —Br, —CF₃, —OH, —O—(C₁-C₁₂)-alkyl, —O—(C₃-C₁₂)-cyclocalkyl, —O—(C₃-C₁₂)-heterocycloalkyl, —NH₃, —NH—(C₁-C₁₂)-alkyl, —NH—(C₃-C₁₂)-cyclocalkyl, —NH—(C₃-C₁₂)-heterocycloalkyl, —COOH, —CONH₂, —CONH(C₁-C₁₂ alkyl), —CON(C₁-C₁₂ alkyl)₂, —COO(C₁-C₁₂ alkyl), —CO(C₁-C₁₂ alkyl), —SH, —SO₃H, —CN, -alkyl, -acyl;
X and Y are independently selected from —O, —NH, —S, —N—(C₁-C₃₀)-alkyl, - or —(C₁-C₃₀)-alkyl.

In another embodiment the active component is a derivative of 5, 7, 3′, 4′-tetrahydroxyflavan-3-ol C₄-C₈ dimers according to Formula III, or a pharmaceutically relevant enantiomer, salt, or solvate thereof. Formula III is as follows:

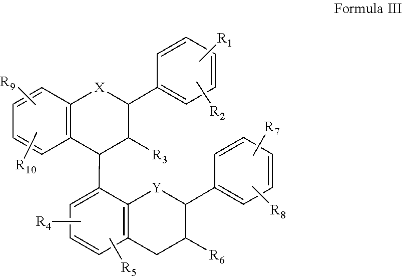

R₁, R₂, R₃, R₄, R₅, R₆, R₇, R₈, R₉ and Rio are independently selected from —H, —F, —Cl, —Br, —CF₃, —OH, —O—(C₁-C₁₂)-alkyl, —O—(C₃-C₁₂)-cyclocalkyl, —O—(C₃-C₁₂)-heterocycloalkyl, —NH₃, —NH—(C₁-C₁₂)-alkyl, —NH—(C₃-C₁₂)-cyclocalkyl, —NH—(C₃-C₁₂)-heterocycloalkyl, —COOH, —CONH₂, —CONH(C₁-C₁₂ alkyl), —CON(C₁-C₁₂ alkyl)₂, —COO(C₁-C₁₂ alkyl), —CO(C₁-C₁₂ alkyl), —SH, —SO₃H, or —CN;
X and Y are independently selected from —O, —NH, —S, —N—(C₁-C₃₀)-alkyl, - or —(C₁-C₃₀)-alkyl.

In one embodiment, the composition contains 0.001% to 50% of 5,7,3′,4′-tetrahydroxyflavan-3-ol C₄-C₈ dimers or derivatives thereof according to Formula I, Formula II, or Formula III. In some embodiments, the composition contains 0.001%, 0.01%, 0.1%, 1%, 5%, 10%, 20%, 30%, 35%, 40%, 45%, or 50% of 5,7,3′,4′-tetrahydroxyflavan-3-ol C₄-C₈ dimers or derivatives thereof according to Formula I, Formula II, or Formula III. The preferred dose of 5, 7, 3′, 4′-tetrahydroxyflavan-3-ol C₄-C₈ dimers or derivatives thereof according to Formula I, Formula II, or Formula III is 30 mg (20% of the extract).

ii. Flavan-3-ol Metabolites

In one embodiment, the composition includes flavan-3-ol metabolites such as 2-(3,4-dihydroxyphenyl)acetic acid and 5-(3,4-dihydroxyphenyl)-gamma-valerolactone. In some embodiments, the composition contains 0.001%, 0.01%, 0.1%, 1%, 5%, 10%, 20%, 30%, 35%, 40%, 45%, or 50% flavan-3-ol metabolites. In another embodiment, the metabolite is a monomer of 5,7,3′,4′-tetrahydroxyflavan-3-ol C₄-C₈ dimers or derivatives thereof.

iii. Quercetin

In one embodiment, a second active agent of F. dibotrys extract is quercetin. Quercetin is a flavonoid found in fruits and vegetables such as but not limited to apples, berries, Brassica vegetables, capers, grapes, onions, shallots, tea, and tomatoes, as well as many seeds, nuts, flowers, barks, and leaves. Quercetin is also found in medicinal botanicals, including Ginkgo biloba, Hypericum perforatum, and Sambucus canadensis. The IUPAC nomenclature for quercetin is 3, 3′, 4′, 5, 7-pentahydroxyfivanone. In one embodiment, quercetin has activity against viruses of the Caliciviridae family.

In another embodiment, the disclosed nutraceutical composition contains an effective amount of quercetin. The preferred dose for quercetin is 20 mg/day.

iv. Rutin

In one embodiment, an additional active agent of F. dibotrys extract is rutin. Rutin is a flavonol, abundantly found in plants, such as passion flower, buckwheat, tea, and apple. The IUPAC name for rutin is 3, 3′, 4′, 5, 7-pentahydroxyflavone-3-rhamnoglucoside. Other names for rutin include also called as rutoside, quercetin-3-rutinoside, and sophorin. In one embodiment, rutin has activity against viruses of the Caliciviridae family.

In another embodiment, the disclosed nutraceutical composition contains an effective amount of rutin. The preferred dose for rutin is 20 mg/day.

v. Sapogenin

Another embodiment provides another active agent of F. dibotrys extract. The active agent is Haike sapogenin. Sapogenins are the aglycones, or non-saccharide, portions of the family of natural products known as saponins. They are found in the tubers of various plants. In one embodiment, Haike sapogenin has activity against viruses of the Caliciviridae family.

In another embodiment, the F. dibotrys extract contains an effective amount of Haike sapogenin. The preferred dose for sapogenin is 15 mg/day.

### C. Herbal Compounds

The disclosed nutraceutical compositions of F. dibotrys extract can be combined with an herbal compound or molecule.

In one embodiment, the F. dibotrys nutraceutical composition contains synthetic catechins or polyphenols.

In some embodiments, the F. dibotrys nutraceutical composition contains milk thistle extract. Silymarin is a flavonoid found in milk thistle and is believed to have antioxidant properties. The compositions can contain 140 mg-200 mg milk thistle extract per dose. The preferred dose of milk thistle extract is 150 mg/day. In another embodiment the F. dibotrys nutraceutical can contain synthetic silymarin.

In another embodiment, the F. dibotrys nutraceutical composition is combined with Semen cassia extract. The compositions can contain 0.1 g to 15 g of Semen cassia. The preferred dose of Semen cassia extract is 1 g per day.

In another embodiment, the F. dibotrys nutraceutical composition is combined with Spica prunellae extract. The composition can contain 0.1 g to 15 g of Spica prunellae extract. The preferred dose of Spica prunellae extract is 1 g per day.

In some embodiments, the F. dibotrys nutraceutical composition contains compounds from Radix or Forsythia. Radix angelicae pubescentis and Forsythia suspense are believed to have anti-inflammatory properties. The composition can contain 0.1 g to 15 g of compounds from Radix or Forsythia. The preferred dose of compounds from Radix or Forsythia is 1 g per day.

In another embodiment, the nutraceutical composition contains compounds from Thlaspi arvense Linn., Tai Zi Shen (Pseudostellaria heterophylla), or Poria (Wolfporia cocos). The compositions can contain 0.1 g to 30 g of compounds from Thlaspi arvense Linn., Tai Zi Shen (Pseudostellaria heterophylla), or Poria (Wolfporia cocos). The preferred dose of compounds from Thlaspi arvense Linn., Tai Zi Shen (Pseudostellaria heterophylla), or Poria (Wolfporia cocos) is 1 g per day.

### D. Nutraceutical Compositions

Pharmaceutical compositions containing the F. dibotrys extract can be formulated for administration by enteral routes and can be formulated in dosage forms appropriate for each route of administration. The preferable route of delivery for the nutraceutical composition is orally.

The compositions disclosed herein can be administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed F. dibotrys nutraceutical compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed F. dibotrys nutraceutical composition, generally dosage levels of 0.1 mg to 10 g are administered to mammals. The preferred dose of F. dibotrys extract is 150 mg per dose, twice a day.

i. Formulations for Oral Administration

In some embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712, which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the F. dibotrys nutraceutical composition and inert ingredients which protect the composition in the stomach environment, and release of the biologically active material in the intestine.

In some embodiments, the nutraceutical compositions are formulated in capsules or tablets. The capsules or tablets can contain ground or powdered raw herbs or plants, or dried extract. The nutraceutical compositions can be formulated as a once-daily supplement, a twice daily supplement, or a three times daily supplement.

For oral formulations, the location of release may be the stomach, the small intestine (the duodenum, the jejunum, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™ cellulose acetate phthalate (CAP), Eudragit L™, Eudragit S™, and Shellac™. These coatings may be used as mixed films.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

In another embodiment, the F. dibotrys nutraceutical composition is administered in the form of a whole herb tea. Ground or powdered dried raw herbs or dried powdered extract are processed in a manner such that the extract is released into the boiling water during preparation of the tea.

### III. Methods of Manufacture

Methods of making medicinal plant extracts are well known in the art. Extraction involves removing the medicinally active portion of the plant from the inert components using selective solvents in standard extraction procedures. Plant and herb extracts vary in the solvent used for extraction, temperature, and extraction time. Types of extracts include but are not limited to alcoholic extracts (tinctures), vinegars (acetic acid extracts), hot water extract (tisanes), long-term boiled extract, decoctions, and cold infusion of plants (macerates). Exemplary extraction procedures include but are not limited to maceration, infusion, digestion, decoction, percolation, hot continuous extraction (Soxhlet), aqueous alcohol extraction by fermentation, counter-current extraction, and ultrasound extraction (sonication). The extract obtained from the plant is relatively impure liquid that can be used in the form of tinctures and fluid extracts without additional processing. However, the extract can be processed further to be incorporated into other dosage forms such as tablets or capsules.

### A. Maceration

In one embodiment, the F. dibotrys extract can be prepared using maceration technique of extraction. In this method, the whole or coarsely powdered plant is placed in a stoppered container with a solvent and allowed to stand at room temperature for at least 3 days with frequent agitation until the soluble matter has dissolved. Common solvents that are used to extract bioactive compounds from plants include but are not limited to water, ethanol, methanol, chloroform, ether, and acetone. The mixture is strained, the solid material is pressed, and the combined liquids are clarified by filtration or decantation after standing. Infusions can be prepared by macerating the whole plants for a short period of time with cold or boiling liquid. Infusions are dilute solutions of the readily soluble portions of the whole plant. Digestion is a form of maceration in which low heat is used during the process of extraction. This increases the solvent efficiency.

### B. Hot Continuous Extraction

Plant extracts can be obtained through hot continuous extraction (Soxhlet). Generally, a small amount of dry sample is placed in a thimble. The thimble is then placed in a distillation flask which contains a solvent appropriate for extracting the bioactive of interest. When the liquid content reached the siphon arm, the liquid is emptied into the bottom of the flask. This solution carries extracted solutes into the bulk liquid. The solute remains in the distillation flask and the solvent passes back to the sample in the thimble. The process runs repeatedly until the extraction is completed.

### C. Aqueous Alcohol Extraction by Fermentation

In one embodiment, the herb and plant extracts of the nutraceutical compositions disclosed herein can be produced through aqueous alcohol extraction. This method involves soaking the plant material in solvent for a specified period of time during which it undergoes fermentation. The generation of alcohol in situ facilitates the extraction of the bioactives contained in the plant material.

### D. Ultrasound-assisted Extraction

In one embodiment, the botanical extracts can be obtained through ultrasound assisted extraction (UAE) methods. UAE is a non-conventional mode of extracting bioactives from plants and herbs. In this method, ultrasound waves ranging from 20 kHz to 2000 kHz are pulsed through the intact tissue of the plant or herb. The ultrasound energy causes organic and inorganic materials to leach out of the plant material and into the solvent that the plant is contained within.

### E. Enzyme-assisted Extraction

In another embodiment, the extracts can be obtained through enzyme-assisted extraction. In this method, plants are pre-treated with specific enzymes to facilitate extraction of compounds that are retained in polysaccharides and lipid bodies within the cell wall. Exemplary enzymes include but are not limited to cellulase, α-amylase, and pectinase.

### F. Microwave-assisted Extraction

In some embodiments, botanical extracts are prepared through the method of microwave-assisted extraction (MAE). This method involves extracting soluble products into a fluid form using microwave energy and was first described by Alupului (U.P.B. Sci. Bull., Series B, 74:129-142 (2012)). Microwave energy disrupts hydrogen bonding between molecules, enhancing the migration of dissolved ions out of the plant matrix while also promoting solvent penetration into the plant matrix.

### IV. Methods of Use

Nutraceutical compositions and their use thereof for treating and/or preventing an infection of a subject by viruses of the Caliciviridae family are provided herein. Caliciviridae viruses that infect humans include noroviruses and sapoviruses. The disclosed compositions can be formulated for oral or rectal administration. One embodiment provides nutraceutical compositions for prophylactically or therapeutically treating norovirus and sapoviruses in a subject in need thereof.

In some embodiments, the effect of the nutraceutical compounds and compositions thereof on a subject is compared to a control. For example, the effect of the composition on a particular symptom, pharmacologic, or physiologic indicator can be compared to an untreated subject or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art.

A. Treating Caliciviridae virus infection

Methods of using the disclosed nutraceutical compositions to inhibit viruses of the Caliciviridae family are disclosed herein. Methods typically include administering to the subject an effective amount of a composition including F. dibotrys extract. In another embodiment, an effective amount of a composition including quercetin, rutin, and 5, 7, 3′, 4′-tetrahydroxyflavan-3-ol C₄-C₈ dimers is administered to the subject to inhibit viruses of the Caliciviridae family. The Caliciviridae viruses can be noroviruses or sapoviruses. Inhibition of noroviruses or sapoviruses can occur through but are not limited to the following, inhibition of viral entry and attachment, inhibition of viral replication, or interfering with the viral capsid.

In one embodiment, the disclosed nutraceuticals can inhibit norovirus or sapovirus without negatively affecting the host cells. The disclosed nutraceuticals compositions can be administered to a subject in need thereof orally or rectally for 1, 2, 3 days or more as needed until symptoms of norovirus or sapovirus have subsided. The compositions can be administered 1, 2, 3, or more times per day as needed.

### B. Preventing Caliciviridae Virus Infection

Methods of using the disclosed compositions to prevent, inhibit, or reduce Caliciviridae virus infection are disclosed herein. Methods typically include orally or rectally administering to the subject in need thereof an effective amount of a composition including F. dibotrys extract. In another embodiment, an effective amount of a composition including quercetin, rutin, and 5,7,3′,4′-tetrahydroxyflavan-3-ol C₄-C₈ dimers is administered to the subject in need thereof to prevent infection. Subjects in need of prophylactic care include those who have been exposed to the virus, are suspected of being exposed of the virus, or are in an environment where exposure to the virus is likely. Without being bound by any one theory, it is believed that the disclosed compositions can protect intestinal epithelial cells from viral infection. Prophylactic administration of the disclosed nutraceutical compositions to subjects who are not actively infected with norovirus or sapovirus can prevent them from becoming infected.

The disclosed compositions can be administered prophylactically three times daily, twice daily, once daily, or every other day. In a preferred embodiment, the disclosed compositions are administered twice daily. In one embodiment, the composition is administered to the subject continuously during times of high norovirus or sapovirus activity. In another embodiment, subjects are administered the disclosed compositions on a daily basis for an extended period of time, for example six-months, one-year, two-years, or more than two years.

a. High Risk Subjects

In some embodiments, certain subjects are at a higher risk of being infected with norovirus and sapovirus. Subjects that are at high risk for developing a norovirus or sapovirus infection include but are not limited to people 65 years and older, pregnant women, young children, and people with certain chronic medical conditions such as asthma, diabetes, or heart disease. These high risk subjects are not only more likely to become infected with these viruses, but are also more likely to suffer from complications from the viruses. Examples of norovirus related complications include but are not limited to severe dehydration, malnutrition, and death. Complications can result in hospitalization, and in some cases, death. In one embodiment, high risk subjects can be administered the disclosed compositions to prophylactically treat norovirus or sapovirus infection.

In one embodiment, high risk subjects are continuously administered the disclosed nutraceutical compositions during times of high norovirus activity. The high risk subjects can be administered the disclosed compositions 1, 2, 3, or more times daily.

### C. Improved Intestinal Health

In some embodiments, the disclosed nutraceutical compositions can reduce intestinal symptoms of norovirus and sapovirus infection. Without being bound to any one theory, it is believed that the disclosed nutraceutical compositions can reduce inflammation in the intestines, thus reducing symptoms of viral infection. In one embodiment, the antioxidant properties of 5, 7, 3′, 4′-tetrahydroxyflavan-3-ol C₄-C₈ dimers can reduce inflammation in damaged or infected intestine. Flavan-3-ols have been shown to behave as antioxidants via several mechanisms including the scavenging of free radicals. The generation of free radicals can cause oxidative damage to DNA, lipids, and proteins, which if left unchecked can lead to disease progression. In one embodiment, the disclosed nutraceutical compositions can act as antioxidants to reduce levels of free radicals and subsequent inflammation.

The disclosed F. dibotrys nutraceutical compositions can be administered to a subject in need thereof to improve or increase intestinal health. Without being bound by any one theory, it is believed that the disclosed compositions promote the growth and repair of damaged intestinal cells.

### EXAMPLES

### Example 1. Pretreatment of CRFK Cells with Different Concentrations of XC for 1 h Before TCID50 Assay

### Materials and Methods

Cat kidney CRFK cells were plated in 96-well tissue culture plates in MEM culture medium with fetal bovine serum and antibiotics at 37° C., 5% CO₂. When cells covered the surface of each well to >90%, F. dibotrys extract (referred to as XC throughout the Examples) dissolved in MEM culture medium (filtered) was added at 0, 0.1%, 0.2% and 1%, followed by incubation for 1 hour. XC medium was then removed and feline calcivirus strain 9 (FCV F9) virus was added at different dilution concentrations from 10⁻⁵ to 10⁻⁸. After 1 hour absorption, the virus was removed and cell growth medium was added to each well. Results were recorded and calculated after 5 days.

### Results

FIG. 1 demonstrates results from 3 independent experiments that indicate a significant inhibition of viral infection at all concentrations of XC extract, even though there was no direct contact of XC extract and virus. Specifically, XC extract at all concentrations reduced FCV F9 infection by more than 50% if the extract was incubated with CRFK cells for 1 hour before FCV F9 infection (n=3, p<0.01, two tailed t-test). There is no statistical difference among all concentrations (0.1% and 0.2%, p=1, 1% vs. 0.1 and 0.2%, p=0.42, two tailed t-test).

### Example 2. XC Extract and FCV F9 Virus were Added to Cells at the Same Time

### Materials and Methods

CRFK cells were plated in 96-well plate as described above. XC at 0.1%, 0.2% and 1% were mixed with FCV F9 virus at different dilutions shown above. The mixture was removed after 1 hour absorption, and the mixture was replaced with cell growth medium. Result was recorded and calculated after 5 days.

### Results

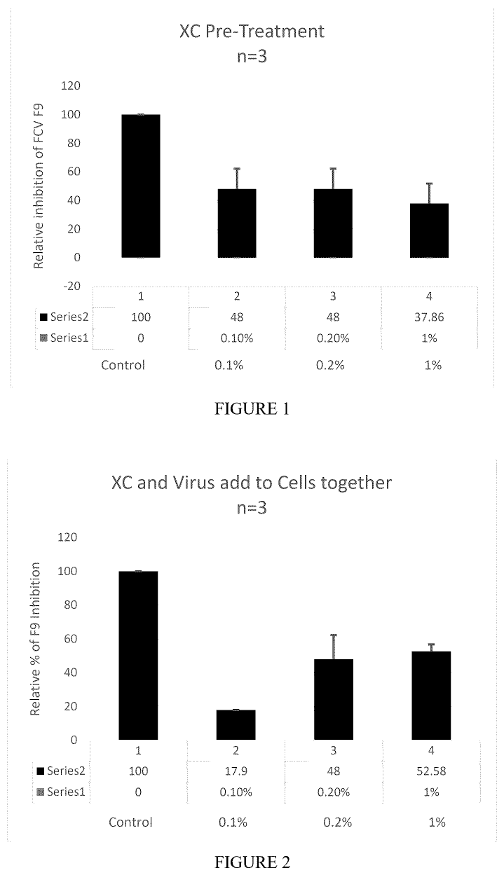

FIG. 2 shows that XC at all concentrations significantly inhibited FCV F9 viral infection in CRFK cells. Data was obtained from 3 independent experiments. XC extract at all concentrations significantly reduced FCV F9 infection (n=3, p<0.01, two tailed t-test). Lower concentrations of XC extract have higher efficacy than higher concentrations. More specifically, 0.1% extract showed higher efficacy (17.9% infection rate) than other concentrations (48% and 52.58% infection rate). Two tailed t-test indicates the differences are statistically significant (p=0.02, p=0.0001). There is no statistical difference between 0.2% and 1%.

### Example 3. XC Extract Added after FCV F9 Viral Infection of CRFK Cells

### Materials and Methods

CRFK cells were cultured as described above. FCV F9 virus was added to the wells at different dilutions and allowed to incubate for 1 hour. FCV F9 virus was removed and XC extract in MEM medium was added to the wells. The cells were allowed to grow for 5 days before result recording was performed.

### Results

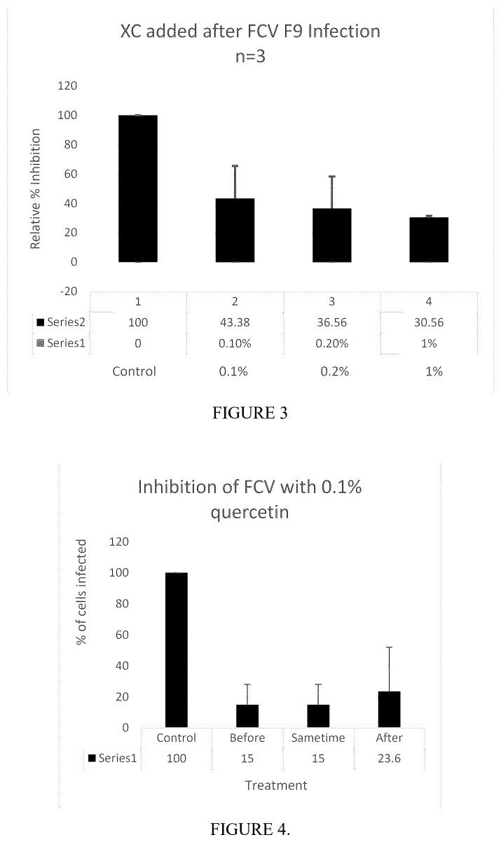

FIG. 3 shows the results from 3 independent experiments demonstrating that without direct contact with the virus, XC extract at all concentrations significantly reduced FCV F9 infection by more than 3 fold. Specifically, XC extract at all concentrations significantly reduced FCV F9 infection (n=3, p<0.001). There is no statistical difference among the concentrations (p=0.72, p=0.37, p=0.65, two tailed t-test).

In conclusion, FCV F9 virus, a surrogate of human norovirus that is resistant to alcohol, can be effectively inhibited by XC extract using different incubation methods. The methods limited the use of XC extract to just once and the results are significantly better than 70% alcohol. FCV F9 is one of the most difficult viruses to inactivate due to its size and non-enveloped structure similar to poliovirus. Thus, XC is a strong inhibitor of FCV F9 virus.

### Example 4. Effect of Quercetin on FCV F9 Infection

### Materials and Methods

50 μl of the virus was added to 450 μl HBSS making a 10⁻¹ viral titer mix. A series of dilutions made from this mix up to 10⁻⁸. Virus was added to all wells by loading 250 μl from each dilution to the designated wells, same as for viral titer. 3 well repeats were prepared. Quercetin was prepared at 10% quercetin in DMSO.

For experiments in which quercetin was added before viral infection, the quercetin was diluted to 0.1% using MM (1:100), added to cells, and incubated for 1 hour.

For experiments in which quercetin was added at the same time as viral infection, the virus was diluted in 0.1% quercetin MM/DMSO, added to wells, incubated for 1 hour, and then MM was added.

For experiments in which quercetin was added after viral infection, viral dilutions were added to the cells, incubated for 1 hour, then 0.1% quercetin in MM/DMSO was added. The medium was changed to MM after 60 minutes.

### Results

As shown in FIG. 4, quercetin significantly inhibited FCV F9 infection of CRFK cells either before or after the virus infected cells, and also if the quercetin is added with the virus for infection.

### Example 5. Effect of Rutin on FCV F9 Infection

### Materials and Methods

50 μl of virus was added to 450 μl HBSS to create a 10⁻¹ viral mix. A series of dilutions made from this mix up to 10⁻⁹. 100 μl from each dilution was added to the designated wells. 4 well repeats were prepared. Rutin was prepared at 10% rutin in DMSO as stock.

For experiments in which rutin was added before viral infection, the rutin was diluted to 0.1% using MM (1:99), added to cells and incubated for 1 hour.

For experiments in which rutin was added simultaneously with virus, the virus was diluted in 0.1% rutin MM/DMSO, the mixture was added to wells, incubated for 1 hour.

For experiments in which rutin was added after viral infection, viral dilutions were added to the cells, incubated for 1 hour, 0.1% rutin MM/DMSO was added to the cells. The medium in the wells was changed to MM after 60 minutes.

### Results

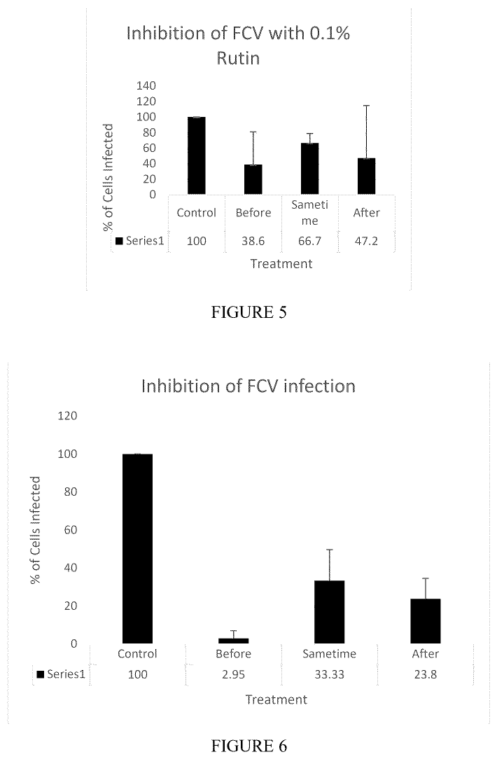

As shown in FIG. 5, rutin inhibited FCV infection when it was added before, after, or at same time as the virus. On the other hand, the inhibition effect is weaker compare to quercetin. This may due to the glycosidic modification.

### Example 6. Effect of 5, 7, 3′, 4′-Tetrarydroxyflavon-3-Ol C₄-C₈ Dimers on FCV F9 Infection

### Materials and Methods

50 μl of virus was added to 450 μl complete EMEM to create a 10⁻¹ viral mix. A series of dilutions made from this mix up to 10⁻⁸. Virus was added to all wells by loading 250 μl from each dilution to the designated wells. 3 well repeats were prepared. Purified 5, 7, 3′, 4′-tetrarydroxyflavon-3-ol C₄-C₈ dimers was diluted to 0.1% in complete EMEM.

For experiments in which 5, 7, 3′, 4′-tetrarydroxyflavon-3-ol C₄-C₈ dimers was added before the virus, 5, 7, 3′, 4′-tetrarydroxyflavon-3-ol C₄-C₈ dimers was diluted to 0.1% using MM (1:100), added to cells and incubated for 1 hour before the virus was added to the cells.

For experiments in which 5, 7, 3′, 4′-tetrarydroxyflavon-3-ol C₄-C₈ dimers was added simultaneously with the virus, the virus was diluted in 0.1% purified complete EMEM, added to wells, incubated for 1 hour, then complete EMEM was added to the wells.

For experiments in which 5, 7, 3′, 4′-tetrarydroxyflavon-3-ol C₄-C₈ dimers was added after viral infection, viral dilutions were added to wells, incubated for 1 hour, 0.1% complete EMEM was added to the wells. The medium was changed to complete EMEM after 60 minutes.

### Results

FIG. 6 demonstrates that 5, 7, 3′, 4′-tetrarydroxyflavon-3-ol C₄-C₈ dimers is effective against FCV infection comparable to quercetin and better than rutin.

### Example 7. Effect of a Combination of 5, 7, 3′, 4′-Tetrarydroxyflavon-3-Ol C₄-C₈ Dimers and Quercetin on FCV F9 Infection

### Materials and Methods

50 μl of virus was added to 450 μl HBSS to prepare a 10⁻¹ viral mix. A series of dilutions made from this mix up to 10⁻⁸. The virus was added to all wells by loading 100 μl from each dilution to the designated wells. 4 well repeats.

0.1% quercetin and 0.1% 5, 7, 3′, 4′-tetrarydroxyflavon-3-ol C₄-C₈ dimers were mixed together in 2% FBS DMEM and filtered.

Viral dilutions were added to the wells. After an hour, 0.1% of the quercetin and 5, 7, 3′, 4′-tetrarydroxyflavon-3-ol C₄-C₈ dimers mixture was added to the cells. The medium was changed to MM after 60 min.

### Results

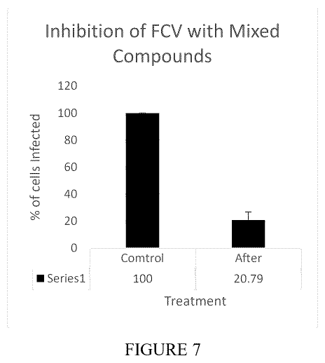

FIG. 7 demonstrates that a mixture of two compounds significantly inhibited FCV infection with very small variation (SD 1.41). This is significantly more consistent than either quercetin or 5, 7, 3′, 4′-tetrarydroxyflavon-3-ol C₄-C₈ dimers alone.

In summary, the purified compounds all have inhibitory effect against feline calicivirus, a surrogate for human norovirus. A mixture of quercetin and 5, 7, 3′, 4′-tetrarydroxyflavon-3-ol C₄-C₈ dimers protected 80% of cells from FCV infection after 1 hour of viral infection. It is possible to treat norovirus infection after an individual is infected with norovirus by multiple oral applications.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for inhibiting or reducing symptoms of viral gastroenteritis in a subject comprising:
administering to the subject a composition comprising Fagopyrum diboyto extract and a pharmaceutically acceptable excipient in an amount effective to inhibit viral gastroenteritis in the subject.

2. The method of claim 1, wherein the viral gastroenteritis is caused by norovirus or sapovirus.

3. The method of claim 1, wherein the composition comprises 0.1 mg to 10 g of Fagopyrum dibotrys extract.

4. The method of claim 1, wherein the composition further comprises 0.001% to 50% of 5, 7, 3′, 4′-tetrahydroxyflavan-3-ol C₄-C₈ dimers.

5. The method of claim 1, wherein the composition further comprises other herbal compounds, extracts, or molecules.

6. The method of claim 5, wherein the herbal compounds or extracts are selected from the group consisting of milk thistle extract, Semen cassiae extract, Spica prunellae extract, radix, forsythia, Thlaspi arvense Linn, Artemisia capillaris Thunb, Tai zi shen, poria, or a combination thereof.

7. The method of claim 1, wherein the composition is formulated for oral administration.

8. The method of claim 1, wherein the composition is formulated for rectal administration.

9. A method for inhibiting or reducing symptoms of viral gastroenteritis in a subject comprising: administering to the subject a composition comprising 5, 7, 3′, 4′-tetrahydroxyflavan-3-ol C₄-C₈ dimers, quercetin, and a pharmaceutically acceptable excipient in an amount effective to inhibit viral gastroenteritis in the subject.

10. The method of claim 9, wherein the composition inhibits or reduces entry of Caliciviridae family viruses into intestinal epithelial cells of the subject.

11. The method of claim 9, wherein the Caliciviridae family virus is norovirus or sapovirus.

12. The method of claim 9, wherein the composition comprises 0.001% to 50% of 5, 7, 3′, 4′-tetrahydroxyflavan-3-ol C₄-C₈ dimers.

13. The method of claim 9, wherein the composition comprises 0.002 to 20% of quercetin.

14. The method of claim 9, wherein the composition further comprises other herbal compounds, extracts, or molecules.

15. The method of claim 14, wherein the herbal compounds or extracts are selected from the group consisting of milk thistle extract, Semen cassiae extract, Spica prunellae extract, radix, forsythia, Thlaspi arvense Linn, Artemisia capillaris Thunb, Tai zi shen, poria, or a combination thereof.

16. The method of claim 9, wherein the composition is formulated for oral administration.

17. The method of claim 9, wherein the composition is formulated for rectal administration.

18. A method for prophylactically treating viral gastroenteritis in a subject at risk of viral infection comprising: administering to the subject an effective amount of a composition comprising 5, 7, 3′, 4′-tetrahydroxyflavan-3-ol C₄-C₈ dimers, quercetin, and a pharmaceutically acceptable excipient to inhibit or reduce entry of Caliciviridae family viruses into intestinal epithelial cells of the subject.

19. The method of claim 18, wherein the subject in need thereof has been exposed to Caliciviridae family viruses, are suspected of being exposed to Caliciviridae family viruses, or are in an environment where exposure to Caliciviridae family viruses is likely.

20. The method of claim 18, wherein the composition is administered to the subject once daily, twice daily, or three times daily.

21. The method of claim 18, wherein the composition is administered to the subject throughout the duration of the period the subject is at risk for viral infection.

22. A compound according to Formula II 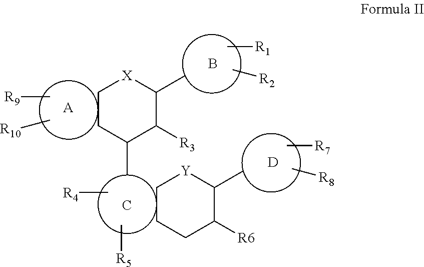
wherein:
rings A, B, C, and D are independently six-membered aryl or nitrogen-containing herteroaryl mono- or bicycylc ring systems containing zero or more nitrogen atoms such as phenyl, pryridine, pyrimidine, pyridazine, pyrazine, triazine, quinolone, quinazoline, isoquinoline, naphthalene, naphthyridine, indole, isoindole, cinnoline, phthalazine, quinoxaline, pteridine, purine, and benzimidazole.
R₁, R₂, R₃, R₄, R₅, R₆, R₇, R₈, R₉ and Rio are independently selected from —H, —F, —Cl, —Br, —CF₃, —OH, —O—(C₁-C₁₂)-alkyl, —O—(C₃-C₁₂)-cyclocalkyl, —O—(C₃-C₁₂)-heterocycloalkyl, —NH₃, —NH—(C₁-C₁₂)-alkyl, —NH—(C₃-C₁₂)-cyclocalkyl, —NH—(C₃-C₁₂)-heterocycloalkyl, —COOH, —CONH₂, —CONH(C₁-C₁₂ alkyl), —CON(C₁-C₁₂ alkyl)₂, —COO(C₁-C₁₂ alkyl), —CO(C₁-C₁₂ alkyl), —SH, —SO₃H, or —CN.
X and Y are independently selected from —O, —NH, —S, —N—(C₁-C₃₀)-alkyl, - or —(C₁-C₃₀)-alkyl,
or a pharmaceutically relevant enantiomer, salt, or solvate thereof.

23. A compound according to Formula II 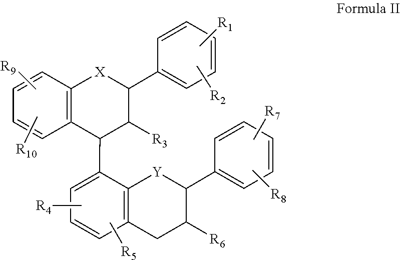
wherein:
R₁, R₂, R₃, R₄, R₅, R₆, R₇, R₈, R₉ and Rio are independently selected from —H, —F, —Cl, —Br, —CF₃, —OH, —O—(C₁-C₁₂)-alkyl, —O—(C₃-C₁₂)-cyclocalkyl, —O—(C₃-C₁₂)-heterocycloalkyl, —NH₃, —NH—(C₁-C₁₂)-alkyl, —NH—(C₃-C₁₂)-cyclocalkyl, —NH—(C₃-C₁₂)-heterocycloalkyl, —COOH, —CONH₂, —CONH(C₁-C₁₂ alkyl), —CON(C₁-C₁₂ alkyl)₂, —COO(C₁-C₁₂ alkyl), —CO(C₁-C₁₂ alkyl), —SH, —SO₃H, or —CN.
X and Y are independently selected from —O, —NH, —S, —N—(C₁-C₃₀)-alkyl, - or —(C₁-C₃₀)-alkyl,
or a pharmaceutically relevant enantiomer, salt, or solvate thereof.

24. A composition comprising:
one or more of 5, 7, 3′, 4′-tetrahydroxyflavan-3-ol C₄-C₈ dimers according to Formula I, Formula II, or Formula III,
quercetin,
rutin,
sapogenin,
and optionally a pharmaceutically acceptable excipient.

25. The composition of claim 24, wherein the composition comprises 1 mg-75 mg 5, 7, 3′, 4′-tetrahydroxyflavan-3-ol C₄-C₈ dimers, 20 mg quercetin, 20 mg rutin, and 15 mg sapogenin.
